(19) United States
(12) Patent Application Publication
(10) Pub. No.: US 2025/0025414 A1
(43) Pub. Date: Jan. 23, 2025

(54) READY-DELIVERY ORAL DOSAGE FORM FOR LABORATORY ANIMALS

(71) Applicant: Duquesne University of the Holy Spirit, Pittsburgh, PA (US)

(72) Inventors: Ira Shea Buckner, Pittsburgh, PA (US); Rehana Khan Leak, Pittsburgh, PA (US); Aleem Gangjee, Allison Park, PA (US); Anuj Sunil Jamenis, Maharashtra (IN)

(73) Assignee: Duquesne University of the Holy Spirit, Pittsburgh, PA (US)

(21) Appl. No.: 18/774,109

(22) Filed: Jul. 16, 2024

Related U.S. Application Data
(60) Provisional application No. 63/527,213, filed on Jul. 17, 2023.

(51) Int. Cl. A61K 9/00; A61K 47/38; A61K 47/42

(52) U.S. Cl. CPC A61K 9/0056; A61K 47/38; A61K 47/42

(57) ABSTRACT

The present invention is a ready-delivery oral dosage form for laboratory animals, for the testing of candidate active agents, that meets a number of design parameters. First, the oral dosage form is chewable but neither fragile nor sticky, so it does not fall apart or scatter in the living space in which it is administered—but also cannot become lodged in the animal's teeth or mouth. Second, the palatability profile is greater than the ordinary animal feed (such as rodent chow and so forth) and therefore will be consumed immediately and preferentially, when administered, prior to any other available food. Finally, the oral dosage form must be readily compounded such as with an automated tablet press without the need to add heat or volatile solvents, with inclusion of active agent added followed by optional refrigeration prior to administration.

### STATEMENT OF FEDERALLY SPONSORED RESEARCH

This invention was made with government support under Contract R21AG068608 awarded by The National Institutes of Health. The government has certain rights in the invention.

### FIELD OF THE INVENTION

The present invention pertains to a specialty ready-delivery animal laboratory oral dosage form, which avoids invasive dosing methods, and which also prevents or minimizes waste, active agent deterioration, and imprecise dosing.

### BACKGROUND OF THE INVENTION

Pre-clinical drug trials typically involve animals, often to evaluate the relationship between the dose of an experimental compound and pharmacologic effects. The quality of these studies can be dramatically reduced if the dosing lacks precision. Drug doses can be administered parenterally, but such procedures are labor intensive and inconvenient—and stressful for the animals. The active agent can be dissolved in a liquid vehicle that is administered by forced oral gavage, or mixing of the drug with (say) rodent chow—inasmuch as merely adding the active agent to drinking water does not assure correct dosing at all, and rodents and other laboratory animals do not know how to swallow human-type oral dosage forms “whole.” Of course, not just any new compounds readily dissolve in liquids that rodents can safely drink. Moreover, even compounds that can be dissolved at practical concentrations often taste unpleasant enough for animals to limit their fluid intake, so at-will fluid dosing is largely impractical, additionally because liquid-based excipients can often cause undesirable reactivity, with consequent instability of the active agent. Mixing the drug with food instead of liquid eliminates certain problems, but it remains difficult to control and to measure accurately how much of the drug the animals have ingested, particularly because laboratory animals commonly grind and strew their chow pellets around their living spaces. With unwanted scattering of medicated traditional pellets, accurate dosing is untenable. Finally, sensitive or volatile active agents ought not to be left at ambient temperatures in an animal dwelling enclosure for any extended period, during which time pharmacologic deterioration can occur. All these dosing issues pose a serious impediment to pre-clinical work on animals and limit their clinical translatability. In order to solve these and other issues, a need remains for a ready-delivery oral dosage form for laboratory animals, suitable for delivery of virtually any active agent intended for oral administration. This need persists among all laboratory animals, including without limitation mice, rats, reptiles, cats, rabbits, pigs, dogs, ferrets, guinea pigs, monkeys and any other laboratory animal for which consistent and precise active agent dosing is needed.

### SUMMARY OF THE INVENTION

In order to meet this need, the present invention is a ready-delivery oral dosage form for laboratory animals that meets a number of design parameters. First, the oral dosage form is chewable but neither fragile nor sticky, so it does not fall apart or scatter in the living space in which it is administered—but also cannot become untenably lodged in the animal's teeth or mouth. Second, the palatability profile is greater than the ordinary animal feed (such as rodent chow and so forth) and therefore will be consumed immediately and preferentially, when administered, prior to any other available food. Finally, the oral dosage form must be formulated with little more than an automated tablet press, with inclusion of active agent added followed by optional refrigeration prior to administration, but importantly without the necessity of adding heat or volatile solvents during tableting. The dosage forms themselves assure precise dose administration, and the refrigeration minimizes or eliminates deterioration or denaturation of the active agent such as might occur at ambient temperature. The present dosage form is designed for ready compounding of investigational (candidate) drugs and active agents, and is not intended for creating shelf-stable products for use at a significantly later time. The invention embraces the ultimate dosage form itself (and the method of administering it) and the convenient method of making it with a pre-mix of the necessary excipients and diluents. When ready-delivery dosage forms of the present invention are prepared and provided to a laboratory animal, the animal will usually if not virtually always consume all of it within 15-25 minutes, making dosing tracking both convenient and precise.

### DETAILED DESCRIPTION OF THE INVENTION

The idea of “a chew” dosage form, to administer a pharmaceutical to an animal (or human), is certainly not new. Many canine monthly pharmaceuticals, such as flea-and-tick or heartworm medications, can be readily obtained at present in a flavored-chew form, often “bacon” flavored, for example. Indeed, chewable dosage forms such as gummies are common for people, too, from vitamins to nutritional supplements to, in some cases, even traditional pharmaceutically active agents. The present invention, however, addresses a completely different paradigm and a heretofore unmet need-precise dosing of one or more candidate active agents, to laboratory animals, in a dosage form (and method) much more specialized than merely “a chew.” Off-the-shelf formulations of widely prescribed chewable dosage forms for animals do not meet this need, or even address the problem which the present invention solves—as discussed herein.

Most of the time, the ready-delivery dosage forms of the present invention will be compounded by the investigators in or near the animal laboratory. For pre-clinical testing of new and candidate active agents, by definition there will not already be available commercial sources for previously formulated drug. Even if there were, human dosage forms are not generally useful in animals anyway, as explained above. The present invention therefore provides a particularly designed pre-mix of materials and excipients that can be compounded together with the desired, carefully measured dose of active agent, for administration to a laboratory animal. The pre-mix critically contains an ingredient profile that in turn creates a dosage form that a laboratory animal can bite and chew but that is not fragile, that is, does not break apart or scatter significant particles or dust when handled, but is not too sticky to get stuck in the mouth or teeth. As described further below, “not fragile” embodies certain combined objective friability and tensile strength parameters that together keep the dosage form from scattering, while the material remains readily chewable and consumable by the laboratory animal. This same ingredient profile also creates a palatability profile, so that in turn the compounded medicament will be preferentially consumed by the laboratory animal before any other available food. Finally, once again the ingredient profile lends itself to ready compounding—importantly, without the necessity of adding heat or volatile solvents during the process—so that the active agent under investigation can be included in the desired/required doses, and the compounded material and active agent kept under refrigeration, if desired, prior to administration. For laboratories where personnel wish to prepare their own ready-delivery dosage forms, no extensive special equipment is necessary other than a pill press or automated tablet press, or the methods discussed herein may be readily executed by any including small contract pharmaceutical laboratories who conduct compounding according to the present invention, on demand. When animal laboratories and pharmaceutical installations form part of the same university or other entity, the present technology can create a synergy between these animal and pharmacy departments. It should also be noted, as discussed further below, that the 17% (maximum) friability tested for the present dosage refers specifically to more intense stresses associated with manufacturing operations like coating, where the tablets are tumbled in a drum as they are sprayed with coating solution, or packaging and shipping where the tablets might be exposed to shock from being dropped or rattling around inside a bottle. Friability determinations are conducted according to standardized testing protocols. The present dosage form's maximum 17% average maximum friability is an empirical value that is meant to correlate very roughly with the risk of a tablet's losing a significant piece at some point in its life, after compression, under commercial circumstances that do not apply here. Under the use conditions envisioned, the tablets have been observed to have lost much less material in the animal's living area than during the standard friability test. For most or all of the presently contemplated uses of the present dosage forms, tableting or compounding occurs so near the animal living space—without stresses such as additional coating tumbling, packing, or “bumpy” transport by common carrier—that the present dosage form disintegration will be or approach zero most or all of the time. While the friability parameter of the present inventive dosage forms is not particularly low as determined by the standardized testing, the combination of limited friability (maximum 17%) with the stated tensile strength (infra.) together contribute to a dosage form which is “neither fragile nor sticky,” as referenced elsewhere in this description.

Further according to the above, while the present pre-mix and dosage forms will have formulations and ingredients such as are discussed in detail, below, the key features of the edible dosage form itself (and its pre-mix) are that, when compounded together with active agent and any applicable optional liquid, the compounded dosage form has a tensile strength between 0.2 and 1.5 MPa and a friability between 0 and 17%, according to USP <1216> Tablet Friability. The possible 0 value for friability is a positive assertion, because it represents a dosage form which does not disintegrate at all despite rough handling. For precise dosing and tracking, the most central feature of the present invention is adherence to the tensile strength and friability parameters together, so that there is simultaneously neither wastage nor animal distress (dosage form does not lodge in the teeth or the mouth) and complete and successful consumption of the entire dosage form can be visually confirmed. Those skilled in the art, knowing of the constraints of both tensile strength and friability, will be able to vary the disclosed formulas herein to achieve these two recited parameters together, and discussion appears further below regarding the inclusion of an “anti-friability” binder in the dosage form. In general, the best ingredients (but not the only ones) to use to adjust both tensile strength and friability include gelatin, collagen, food grade cellulose, carrageenan, guar gum, xanthan gum, and their excipient coordinating ingredients such as stearic acid, magnesium stearate, silicon dioxide, potassium sorbate and titanium dioxide. The pre-mix of ingredients is designed so that it can be pressed or compounded at ambient temperatures after adding the correct dose of active agent per unit dosage form, without the necessity of adding either heat or volatile solvents. Typically the pre-mix contains particulated ingredients in which the particle size is <500 microns, and the particles will compound to a dosage form with, when desired, compaction pressure between 5-600 MPa with or without adding additional minor amounts of excipient liquid including but not limited to water. Further as to particle size and particle size distribution, more than 93.6% of the particles are sized within the range 75-500 microns prior to compression and less than 0.2% of the particles were smaller than 75 microns while about 6.2% were larger than 500 microns. Some active agents would not be compatible with a water addition, and the present pre-mix does not require water for compounding. Any water addition occurs prior to tablet compression.

The present ingredient profile will include, in general, all of proteins, starches, fats and natural sugars, with one or more aromatic compounds' being included in at least one of those ingredient categories. By “aromatic” is meant a fraction that creates a pleasing or enticing scent from the perspective of a laboratory animal. A material that contains natural sugars with aromatic compounds could include, for example, dehydrated banana chips or flakes in particulated form, with their distinctive aromatic fraction and associated scent, but many aromatic/sugar equivalents are available such as citrus chips, citrus peels, sweet potato chips or flakes, apple flakes, pineapple flakes, maple syrup, corn syrup, and so on. Starch components can be, without limitation, ground corn, corn grits, oatmeal, cornstarch, soybeans, soy grits, potatoes, barley, sunflower seeds, sesame seeds, wheat germ, cream of wheat, and the like. Fats can include refined cholesterol, pork belly, beef fat, vegetable oils, fish oil(s) such as cod liver oil, or dairy or nut butters, or seed oils including but not limited to canola oil, olive oil, peanut oil, and corn for example. Protein sources can include animal meat, vegetable protein, tofu, dairy proteins, brewer's yeast, nuts and legumes such as soybeans, chickpeas, kidney beans, pinto beans and so forth. (As is apparent, many ingredients can contribute to more than one nutrient category.) A critical formulation component, added together with at least some or all of protein, fat, starch and sugar (having at least one aromatic compound associated with one of those ingredients) is the inclusion of an anti-friability binder. The anti-friability binder can be silicated cellulose, preferably powdered food grade cellulose, or powdered or granulated vegetable gums including but not limited to xanthan gum, guar gum or carrageenan, or gelatin or any other excipient or binder that promulgates anti-friability without creating excess stickiness (mirrored in tensile strength). The presence of the anti-friability binder in the compounded dosage form assures that the animal will be able to chew the dosage form but that the form will not be too friable so as to fall or scatter about the cage or other dwelling enclosure. The importance of the one or more aromatic compounds is twofold—to provide an enticing aroma to the laboratory animal to encourage the dosage form's prompt consumption, and also to provide a masking agent, to mask any unpleasant or bitter taste that would otherwise become prominent in the dosage form, due to the presence of the active agent. Overall, the constituency of the nutritive components of the present dosage form pre-mix will be about ⅓ fat, ⅓ protein, ⅓ carbohydrate, with the latter's being relatively more predominantly simple sugars rather than more complex carbohydrates, although both are generally present. The ideal carbohydrate content of the present dosage form is between 50-70% by weight simple sugars and 30-50% complex carbohydrates.

In addition to the aromatic component of the present invention, the formulations also may incorporate a bitterant in a way that gives new and unexpectedly improved results. We added a bitterant to the formulation and, surprisingly, the presence of the bitterant increased the percent consumption of the present tablets compared to when the bitterant was absent. The pellets containing the bitterant were completely consumed 97% of the time, which represented a significant increase in consumption compared to tablets without the bitterant. We had initially tried adding some bitterant to the formulations to approximate what the dosage form would taste like were it to contain a bitter active agent, and we had no reason to believe its presence would increase overall consumption. After the first week, we introduced a bittering agent and increased the concentration over time. With the bitterant included, the tablets were completely consumed with few exceptions, leading to the complete consumption 97% of the time. The bitterant initially used was denatonium benzoate, which is often added to antifreeze, or nail polish, to make what contains it unpleasant tasting to humans or pets. Many consider denatonium benzoate to be the bitterest substance known to man. Denatonium benzoate is so bitter, when it is used as an adversive additive it will be perceived as bitter at a concentration of 1-10 ppm, and a liquid containing it is considered to be undrinkable (by humans or household pets) at 30-100 ppm. However, we found that the mice vigorously consumed the tablets when they contained anywhere from 10-160 ppm, with the higher range inclusion of 100-160 ppm representing a bitterant additive amount exceeding what is typically or ever added to discourage human or pet consumption. In other words, we found that the mice were not deterred in the least by the presence of the denatonium benzoate at higher levels than typically used as an aversive, and in fact the presence of the denatonium benzoate caused more ready consumption of the tablets than when the denatonium was absent. The 10-160 ppm concentration was equivalent to 3-48 micrograms per 300 milligram tablet. Ironically, among various products that bitterants have been added to in the past include—rat poison. However, typically about 10 ppm maximum of denatonium benzoate have been added to rat poison to make it unappealing to humans or pets, whereas in the present invention we have identified, as set forth above, that 10-160 ppm denatonium benzoate increased the rodent (mouse) consumption of the tablets containing it. Alternatives to denatonium benzoate include denatonium chloride and denatonium saccharinate. Alternative non-denatonium bitterants, which require proportionately greater inclusion due to the relatively lesser bitterness associated with them, include without limitation sucrose octaacetate, quassin, quinine, eucalyptus oil, menthol oil, and citrus oil. Notable, the bitterants of the present invention are not intended to create any sort of burning sensation, so capsicum, capsaicin, or vinegar in strong concentrations are not contemplated as the present aversive agent, which should instead be a true bitterant. Bitterants of this classification are well known in the industry, and it is their inclusion in a laboratory animal pellet or tablet, here, which gives new and unexpectedly improved dosage form consumption results, throughout the entire bitterant additive class.

Food-grade color, ideally blue or green, may be added to the present pre-mixes of ingredients, so that the compounded dosage form has a distinctive color throughout. A colored oral dosage form makes it easier for the investigators to track where and when the dosage forms have been consumed, inasmuch as the color creates a sharp contrast to the pale-hay or yellow or orange color of typical laboratory bedding material. Color has been known for use in prior art versions of medicaments added to animal feed for tracking or shredded or wasted portions, or fecal fractions, but in the case of the present invention the color provides a readily visible toggle—either the dosage form has been consumed, or if not the remaining colored material can be readily collected and quantified. Of all the possible colors, for laboratory use the color blue is the best choice.

Further regarding the cellulose (or vegetable gum) anti-friability component of the present invention, food grade cellulose, microcrystalline cellulose, methylcellulose, or cellulose gum or cellulose gel are interchangeably useable in the present pre-mix formulations, intended for compounding by laboratory personnel with the desired active agent dose. Typically, when cellulose is incorporated in the present dosage forms, the dry-weight cellulose component of the present pre-mix will be about 50 percent by weight. In general, for the alternate anti-friability components, the amounts by weight will be on the order of about 55-65 percent by weight of any other vegetable gums, gelatin or other binders that are substituted for the cellulose component.

The benefit of a ready-delivery agent is not just to the laboratory animal and the investigators—rapid tablet consumption is directly translatable to human oral intake of drugs in pill form, leading to pulsed drug delivery, followed by absorbance through the gastrointestinal tract into the bloodstream. The benefits of this form of direct/rapid oral administration mean that all of the hepatic first-pass effect, blood protein-binding potential and biodistribution from blood into tissues (nervous system, etc) will be maximally analogous to what will happen, clinically, when the same active agent is later orally dosed to human subjects. This means that there are myriad research benefits to the present ready-delivery dosage forms besides the already-important precise dosing and convenience benefits discussed above.

A typical pre-mix for the present invention, as but one example among many, is an admixture of powdered oatmeal, wheat germ, brewer's yeast, cod liver oil, corn syrup, microcrystalline cellulose, colloidal silica and magnesium stearate, to create a “moist” pre-mix that can be compounded, with pressing, with an added active agent measured as to each unit dosage form, As a single example, a pre-mix embodiment can contain 50 g powdered oatmeal, 5 g brewer's yeast, 10 mg dehydrated banana flakes, 94 g. powdered cellulose, 15 g. wheat germ, 4 g. corn syrup, 3 g. magnesium stearate, and 7 grams powdered gelatin. With all these ingredients' being in powdered form as a pre-mix, a portion of the pre-mix may be mixed with a measured dosage of active agent and pressed together to make an oral dosage form with the friability and tensile-strength characteristics described above. The invention inheres in all of the final dosage form, the method of making it and the method of dosing laboratory animals to achieve new and unexpected results. The single example in this paragraph is exemplary only—among the categories of protein, fat, carbohydrate and aromatic compound(s), the substitutions may be virtually endless, as long as the friability and tensile strength features of the invention are maintained. This paragraph does not describe an added bitterant, but a bitterant may be included, of a type and in the amounts disclosed above.

A second exemplary formulation of the present premix, again without limitation, can for example include 25 percent by weight (all percentages are % w/w in the final tablet) oat bran, 10% wheat germ, 5% brewer's yeast, 5% cod liver oil, 5% corn syrup, 49.944-49.960% silicified microcrystalline cellulose, 0.04% Food blue no. 1 aluminum lake and 0-0.016% denatonium benzoate. As variations of this formulation, we also have substituted corn oil for cod liver oil and, also, substituted compressible sugar in place of the corn syrup, in comparable amounts. The mice we observed readily ate these adjusted formulations also. As a more general proposition, the formula contained the following categories of ingredients (again, % w/w in the final tablet): carbohydrates 35-52.5%, yeast 2.75-5%, oil 5-7%, sweetener 5-7%, diluent 29.96-49.96%, pigment 0-0.04% and bitterant 0-0.016%.

Although the invention has been described with particularity above, with reference to particular formulations, amounts, ranges and parameters, the invention is only to be limited insofar as is set forth in the accompanying claims.

We claim:

1. An oral dosage form for a candidate drug for a laboratory animal, comprising a unit dosage amount of one or more candidate active agents, compounded with a particulated pre-mix material containing at least one fat source, one protein source, one carbohydrate source, one aromatic compound, and at least one binder, all of which ingredients adhere under pressure and ambient conditions, without needing added heat or volatile solvents, to make a single unit dosage form which is neither fragile nor sticky.

2. The oral dosage form according to claim 1, wherein said anti-friability component is selected from the group consisting of cellulose, vegetable gum, gelatin, starch, dextrates, dextrin, pregelatinized starch, maltodextrins, compressible sugar, sorbitol, potassium sorbate, lactose, mannitol, sucrose, fructose, calcium phosphate, magnesium carbonate and collagen

3. The oral dosage form according to claim 1, wherein said carbohydrate source is greater than 50% simple sugars and less than 50% complex carbohydrates.

4. The oral dosage form according to claim 1, wherein said single unit dosage form has a friability in the range of 0-17% according to USP <1216> and a tensile strength in the range of 0.2-1.5 MPa.

5. A pre-mix for compounding one or more candidate active agents in or near an animal testing laboratory, comprising an admixture, in particulated form comprising particles, of a fat source, a protein source, a carbohydrate source, and at least one binder, wherein at least one of said fat source, said protein source or said carbohydrate source contains an aromatic compound and wherein a preponderance of said particles are in the size range of 75-500 microns.

6. The pre-mix for compounding according to claim 5, wherein said particles have a particle size distribution wherein 93.6% of the particles are sized within the range 75-500 microns prior to compression, less than 0.2% of the particles are smaller than 75 microns and about 6.2% are larger than 500 microns.

7. A method for compounding one or more candidate active agents for oral administration to one or more laboratory animals, comprising the steps of: a) measuring a precise unit dose of one or more candidate active agents; and b) compounding each precise unit dose at ambient temperature under pressure, without the need to add heat or volatile solvents, with a pre-mix containing a fat source, a protein source, a carbohydrate source, and an anti-friability component, wherein at least one of said fat source, said protein source, or said carbohydrate source contains an aromatic compound.

8. The method according to claim 7 wherein said pre-mix further contains a quantity of a bitterant.
